(19) United States
(12) Patent Application Publication
(10) Pub. No.: US 2014/0210600 A1
(43) Pub. Date: Jul. 31, 2014

(54) METHOD, MOBILE APPLICATION AND MEDICAL SYSTEM TO DETERMINE ASSISTIVE INFORMATION FOR A MEDICAL APPARATUS

(71) Applicants: Stefan Lautenschlaeger, Hausen (DE); Norbert Rahn, Forchheim (DE)

(72) Inventors: Stefan Lautenschlaeger, Hausen (DE); Norbert Rahn, Forchheim (DE)

(21) Appl. No.: 14/168,410

(22) Filed: Jan. 30, 2014

(30) Foreign Application Priority Data
Jan. 30, 2013 (DE) 102013201546.1

(51) Int. Cl. A61B 19/00; G08C 17/02

(52) U.S. Cl. CPC A61B 19/56; G08C 17/02
USPC 340/12.28

(57) ABSTRACT

In a method and system to determine and display assistive information related to the current operating status of a medical apparatus at a mobile auxiliary device, operating parameters describing the current operating status of the medical apparatus are transmitted to the mobile auxiliary device via a wireless (in particular bidirectional) communication connection, and are evaluated at the mobile auxiliary device via a mobile application in order to determine the assistive information, whereupon the assistive information is displayed at least in part by the mobile application.

### BACKGROUND OF THE INVENTION

1. Field of the Invention

The present invention concerns a method to determine and display assistive information related to the current operating state of a medical apparatus. The invention also concerns a medical apparatus, a mobile application and a system for implementing such a method.

2. Description of the Prior Art

In the medical field, a number of medical apparatuses are used that fulfill extremely complex tasks and complicated to operate. For such medical apparatuses it is known to provide operating manuals or the like that explain to an operator how an optimal operation of the apparatus is possible. It is also known to supply operating manuals in electronic form or even to provide them online (thus on the Internet), such that a manner of online help is provided. However, this manner of instructing operators is disadvantageous because it is extremely complicated for the operator to locate the desired information.

It has consequently been proposed to provide a context-sensitive assistance, i.e. to achieve an assistive functionality that considers the current operating state of the medical apparatus. For known medical apparatuses, this is always possible when software is used in order to operate the apparatus and suitable output means are provided to present the assistive information. This means that a context-sensitive assistance functionality in medical facilities must be integrated into the software installed at the medical apparatus for every single medical apparatus.

This solution is complicated to implement and maintain and must be re-implemented, depending on the version, by the manufacturer of the medical device or of the software that runs on the medical device. In addition to this, the output means of many medical apparatuses are not sufficient to reasonably realize such a functionality.

### SUMMARY OF THE INVENTION

An object of the present invention is to provide a context-sensitive assistive functionality that can be operated intuitively, can be serviced well, and can be implemented more simply.

This object is achieved according to the invention by a method to determine and display assistive information related to the current operating state of a medical apparatus at a mobile auxiliary devices, in which method operating parameters describing the current operating state of the medical apparatus are transferred to the mobile auxiliary device via a wireless (in particular bidirectional) communication connection, are evaluated at the mobile auxiliary device via a mobile application to determine the assistive information, whereupon the assistive information is displayed at least in part by the mobile application.

The invention is thus based on outsourcing a context-sensitive assistive functionality that evaluates the current operating state of the medical apparatus to a mobile auxiliary device, preferably a computer designed as a tablet or a mobile telephone, in particular a smartphone. The assistive functionality is thereby realized by a mobile application, thus an application computer program for mobile applications or mobile operating systems. Such mobile applications are frequently abbreviated as “mobile apps” or simply as “apps”. In order to provide the operating parameters describing the current state of the medical apparatus to the mobile auxiliary device (and thus to the mobile application), a wireless communication connection (that is preferably bidirectional) is established between the mobile auxiliary device and the medical apparatus so that the operating parameters can be transferred and be evaluated automatically by the mobile application in the mobile auxiliary device. An interface between the mobile auxiliary device and the medical apparatus (medical technology apparatus) is consequently established via the communication connection. Known communication standards can be used for this purpose, for example Bluetooth or preferably WLAN. Technological limitations (for example a ban on WLAN-capable devices in hospitals) are already frequently being rescinded currently. Hospitals are already known in which an area-spanning WLAN exists.

In summary, instead of the non-context-sensitive online assistance functionality or the operating manuals available as a printed item, the present method provides context-sensitive assistance in the operation of medical apparatuses (for example image acquisition devices; post-processing workstation; signal processing systems; signal detection systems; auxiliary devices such as contrast agent injectors and the like) via a mobile application (“app”) that runs on a mobile auxiliary device such as a tablet or another handheld device.

The invention is based on the insight that mobile applications on mobile auxiliary devices (in particular tablets or smartphones) play an ever increasing role in modern communication and in daily life. Aspects of economic and technological development contribute to the spread of these mobile auxiliary devices with special mobile applications that will also permeate medical technology over time. Young physicians in hospitals tend to expect—based on their everyday experience with mobile applications—that such mobile applications are also provided at their workspace (a hospital, for example).

Such mobile auxiliary devices and mobile applications have the advantage—not only for such persons but also in general—of being very user-friendly and thus intuitively usable. The suitable information is always provided in a simple manner after an evaluation of the current state or the current situation of the medical apparatus (or in general of the clinical workflow). Mobile applications are easy to create and maintain with the use of suitable programmer assistance agents, and the possible assistive information can itself be stored in various databases supplying mobile auxiliary devices, for example, as will be presented in detail in the following.

A platform-independent mobile application that is usable on different mobile auxiliary devices is preferably used. This means that the mobile application can be realized independently of the specifically provided hardware, for example by using operating systems-spanning programming languages, scripting languages and formatting commands. For example, languages such as HTML, JavaScript, PHP and the like can be used. It is not absolutely necessary for the mobile application to be developed by the manufacturer of the medical apparatuses itself; rather, a third party can take over the development of the software (thus of the mobile application). In this embodiment, the mobile application can consequently be used on many different platforms (and consequently mobile auxiliary devices), which improves the usability and reduces the development cost.

In a further embodiment of the present invention, the mobile application is designed to determine and display assistive information at different medical apparatuses, with a setting of the mobile application for a currently operated medical apparatus taking place after transmission of identification information by the medical application via the communication connection and evaluation of the identification information by the mobile application. It is thus particularly advantageous to design the mobile application (and thus the entire method according to the invention) so that context-sensitive assistance not only can be supplied for one medical apparatus; rather, the same mobile application can be used for a larger number of medical apparatuses, such that the development and maintenance cost can be reduced, but at the same time the usage for an operator is simplified, because the operator can use the same mobile application for different medical apparatuses. For this purpose, it is necessary for the medical apparatuses, for which assistive information should presently be determined, to be correctly identified so that the correct assistive functionality is also achieved. For this purpose, identification information is transmitted by the medical apparatus. The identification information can include the precise designation, version and/or serial number, but can also be otherwise designed such that said medical apparatus can be uniquely identified by the mobile application. For example, it is possible to broadcast the identification information essentially together with the operator parameters, or to send the identification information in a targeted manner so that receiving mobile auxiliary devices can be adjusted based on this information.

However, it is possible and preferable according to the invention for the identification information to be requested by the mobile auxiliary device in a request signal via the communication connection. This thus means that, for example, the mobile application can send out a request signal after it is started, and this request signal is accordingly answered by at least one medical apparatus. If necessary, it can be established for which medical apparatuses assistive information is required by evaluating the proximity of medical apparatuses to the mobile auxiliary device. However, it is also conceivable, multiple medical apparatuses transmit identification information for an operator to select those medical apparatuses for which he or she desires assistive information. This is analogously applicable to the case of multiple medical apparatuses sending their identification information unrequested.

In general, within the scope of the present invention it is preferred for every change of an operating parameter to be transferred to the mobile auxiliary device, that a transfer of at least the modified operating parameter to the mobile auxiliary device takes place, wherein the mobile application updates the assistive information upon receipt of modified operating parameters. Messages are thus sent via the interface (realized by the communication connection, which can be designed to be bidirectional) whenever an operating parameter changes, for example if a user interaction or another status change has occurred at the medical apparatus. The current state (status) of the medical apparatus (or of the medical workflow) is thus known to the mobile application, such that context-sensitive assistive information can be determined, for example including text with embedded image material. In this way the actual required information can always be received.

As already noted, in order to determine the assistive information it can appropriately be retrieved using the operating parameters, or query parameters derived from these operating parameters can be retrieved from a database. If assistive information should be updated, for example if a new version of the medical apparatus or the software of the medical apparatus comes into circulation, it is no longer necessary to modify the mobile application itself; rather, it is sufficient to modify, delete and/or supplement assistive information in the database.

The database is itself can be stored on the mobile auxiliary device and/or the database stored on a computer (in particular a server) can be accessed via an additional communication connection (in particular the Internet). It is preferred for the database to be present at an Internet storage site (for example a server). The so-called “cloud” can also advantageously be used as a storage location (“cloud storage”). It is also possible to store the database at least temporarily and/or in part in a corresponding memory device at the mobile auxiliary device.

In a first embodiment, the database is loaded automatically onto the mobile auxiliary device upon start-up of the mobile application. This database, particularly given a mobile application suitable for multiple medical apparatuses, can be related to the currently operated medical apparatus and/or can be a portion of a database including a assistive information for multiple medical apparatuses, this portion being related to the currently operated medical apparatus. This means that at least all portions of the database or databases that are relevant to the currently considered medical apparatus can be stored on the mobile auxiliary device upon starting the mobile application. These portions are advantageously deleted again after ending (closing or exiting) the mobile application. That has the advantage that the assistive information is provided quickly and independently of the additional communication connection or, respectively, its quality. In the event that the mobile application is designed for multiple medical apparatuses, as described above the medical apparatus for which assistive information should be delivered is initially identified, after which the appropriate database, or the appropriate portion of the database, can be retrieved and be kept ready locally.

In an alternative second embodiment, only assistive information related to the current operating state of the medical apparatus is downloaded from the computer. This means that assistive information is retrieved and downloaded from the database only when it is actually required due to the current operating state of the medical apparatus. If the operating state changes, the corresponding assistive information can be deleted again. The storage space requirements at the mobile auxiliary device are markedly reduced in this way.

In another embodiment of the invention, a portion of the assistive information that is to be displayed is selected and displayed based on a user input at the mobile auxiliary device. The user thus can retrieve specified portions of the assistive information via corresponding operation of control elements (in particular a touchscreen). A search function for the assistive information is preferably implemented in this way. This enables terms or defined keywords to be searched for, for example, in order to be able to locate a concrete portion of the assistive information particularly simply and quickly. Known intelligent search functions that, for example, consider preceding queries and/or operating parameters, can also be used in order to increase the user-friendliness and optimally quickly obtain the sought information related to the current operating state of the medical apparatus.

In a further embodiment, assistive information is used that includes at least one operating parameter (in particular at least one operating protocol) for the medical apparatus is used. This means that the assistive information itself can also receive specific operating parameters for the medical apparatus, such that, when these are displayed, a simplified operation of the medical apparatus is possible. Using the displayed operating parameters, it is thus possible to set these even at the medical apparatus.

In another embodiment it is also possible that, upon selection by the user of at least one operating parameter of the assistive information at the mobile auxiliary device, this selection is transferred via the communication connection to the medical apparatus, wherein the at least one operating parameter transferred from the mobile auxiliary device to the medical apparatus is proposed for adjustment and/or is used to control the medical apparatus. In order to further assist the operator, an automatic transfer of operating parameters that should be adjusted anyway (in particular of operating protocols) to the medical apparatus can thus take place because the communication connection is bidirectional anyway. There at the medical apparatus, a suitable additional processing of the operating parameters is conducted, wherein this can optionally be displayed there again for confirmation. If it is confirmed—or immediately, if no suggestion takes place—the at least one transferred operating parameter of the assistive information can be used to control the medical apparatus. A control of the medical apparatus via the assistive functionality presented here is consequently also conceivable to a limited, specifically adjustable degree. The current status of the medical apparatus is analyzed, the matching assistive information is assessed by a user and he can find matching setting suggestions in the assistive information regarding the current operating status of the medical apparatus, and these can be realized automatically.

In this context, it is advantageous for the assistive information to include operating parameters (in particular operating protocols) for different medical matters. This means that suitable operating parameters or operating parameter sets (operating protocols) can be located in the assistive information for different medical circumstances, for example therapy approaches, pathological symptoms and the like. For example, operating protocols can be measurement protocols for image acquisition devices, in particular what are known as organ programs. For other medical apparatuses—for example a contrast agent injector—operating protocols can include parameters of a contrast agent injection and the like. Because the current medical task (and thus the medical circumstance) is known to the operator, it is possible for him or her to suitably use the assistive information without any problems and to extract the optimal operating parameters. Collections known as imaging protocol books can also be made available in a text-sensitive manner at any time in this way. Such imaging protocol books include measurement protocols for different medical circumstances and questions as operating parameters.

It should be noted that the contents of the context-sensitive online help (thus the assistive information) that are described herein can naturally be provided in different languages. If a database is used, for example, the assistive information can be stored therein in a variety of different languages and kept available for the mobile application on the mobile auxiliary device. For example, a specific language for the display of assistive information can take place depending on a general language set at the mobile auxiliary device, for example described by an operating parameter. It is also possible to make this language selection dependent on an identification information of the medical apparatus, via a user prompt for the language of the medical apparatus that results from this. The assistive information itself is preferably provided and monitored for any changes or updates that may be needed by the manufacturer of the medical apparatus. As already noted only the contents of the database require this attention.

If the interface formed by the communication connection is standardized, the mobile application can be provided across manufacturers for a large number of medical apparatuses.

In a further embodiment, the usage of the context-sensitive assistive information described here takes place for a fee. At least one item of usage information related to the determination and/or display of the assistive information, and/or the time of the usage of the mobile application and/or a data set required for the assistive information can be determined by the mobile application, or an additional mobile application and can be evaluated with regard to billing information. For example, the loading processes (in particular number or size) of the assistive information that is used can be recorded and invoiced to the operator. This can also be implemented via the mobile application or another mobile application. The usage information thus does not need to be evaluated in the mobile auxiliary device itself in order to determine the billing information; rather, the billing information can be transferred to an evaluation device for evaluation, in particular to an evaluation server and/or the medical apparatus. There the usage information serves for documentation and/or as a basis for the accounting.

The method according to the invention can be extended by providing support function in the mobile application, that causes at least one item of status information related to the current status of the medical apparatus and/or result information related to a job result of the medical apparatus, to be transmitted via the communication connection (preferably the Internet) to a support computer associated with at least one support employee upon activation of the support function by the user. The mobile application (or an additional mobile application) can thus optionally also be used with the use of a “support” function in order to transmit status information and/or result information to a support team (service coordination center) of the manufacturer of the medical technology apparatus. For this purpose, the data transfer takes place to a support computer (which can be configured as a type of “expert forum”). Support employees or experts in the expert forum can process the support request and deliver assistance to the operator of the medical apparatus for questions that cannot be immediately answered from the assistive information. The status information and/or result information (which can be designated overall as support information) are advantageously accompanied by a request information that reflects the concrete question.

In such a case, command information can be transferred to the medical apparatus upon activation of the support function via the communication connection. A representation of current image data reflecting a display device of the medical apparatus is determined upon receipt of the command information at the medical apparatus, and can be transferred to the mobile auxiliary device, from which the current data are transmitted further as status information. In an embodiment, it is thus possible to transfer “snapshots” or “screenshots” of the display device (a monitor, for example) of the medical apparatus to the mobile auxiliary device since an excellent basis for assessing the entire situation results from this for the support team, or the individual support employees. In addition or as an alternative to such a transfer of image data reflecting a current presentation, at least one acquired image data set is transmitted as result information given an image acquisition device as a medical apparatus. Particularly when a question to a support employee concerns the quality or other properties of an acquired medical image data set, it can be appropriate to also transmit this image data set in order to receive corresponding instructions.

The status information and/or the result information are preferably anonymized and/or encrypted before the transmission to the support computer by the mobile application. This is appropriate since requirements for data protection (in particular that concern patients) can be complied with, and additionally the security of the transfer can be ensured. The information required for the inquiry to a support employee is anonymized and ideally is also transferred with secure encryption.

If a support employee prepares the inquiry, at least one item of response information is transmitted from the support computer to the mobile auxiliary device and displayed there. The mobile application can thus also accept a response present in electronic form and thus bring this to the attention of the operator. Naturally, a support employee can also contact the sender of such an inquiry in another manner, for example by calling (which is advantageous when the mobile auxiliary device has a telephone function).

As already noted, various embodiments of the assistive information are conceivable. In particular, these can include text components and/or images and/or acoustic elements. Images can be embedded into a text and serve for additional illustration of the information included in said text. Acoustic elements are also conceivable, for example speech outputs or comparison sounds that relate to the operation of the medical apparatus.

As already discussed, a mobile telephone (in particular a smartphone) and/or a tablet can be used as a mobile auxiliary device. Tablet computers (frequently also designated as tablet PCs or just tablets) are portable, flat computers that are often designed to be particularly lightweight and have a touchscreen display.

In addition to the method, the invention encompasses a medical apparatus having at least one interface for data transfer to a mobile auxiliary device via a communication connection for implementing the method according to the invention. A medical apparatus according to the invention is thus designed to participate in the method according to the invention in that assistive information for this medical apparatus can be retrieved in a context-sensitive manner via the mobile application. For this purpose, it is necessary for the medical apparatus to transmit the operating parameters to the mobile auxiliary device. For this purpose, the medical apparatus has the aforementioned interface (formed by hardware and software components) in order to enable the transfer to the mobile auxiliary device via the communication connection. The described embodiments of the method according to the invention apply analogously to the medical apparatus according to the invention.

The invention also concerns a non-transitory, computer-readable data storage medium (namely a stored mobile application) that is designed (includes encoded commands) to determine and display an assistive information related to the current operating status of a medical apparatus upon receipt, via a wireless (in particular bidirectional) communication connection, of at least one operating parameter describing the current operating status of the medical apparatus within the scope of the method according to the invention, when the mobile application according to the invention is executed at a mobile auxiliary device. All statements with regard to the method according to the invention apply analogously to the mobile application (“app”) according to the invention, with which the presented advantages can consequently also be achieved.

The invention also encompasses a system that includes a medical apparatus, a mobile auxiliary device and a wireless communication connection between the medical apparatus and the mobile auxiliary device, wherein the system is designed to implement the method according to the invention. All previous statements apply to the system according to the invention.

### BRIEF DESCRIPTION OF THE DRAWINGS

FIG. 1 is a flowchart of an exemplary embodiment of the method according to the invention.

FIG. 2 schematically illustrates the system according to the invention.

### DESCRIPTION OF THE PREFERRED EMBODIMENTS

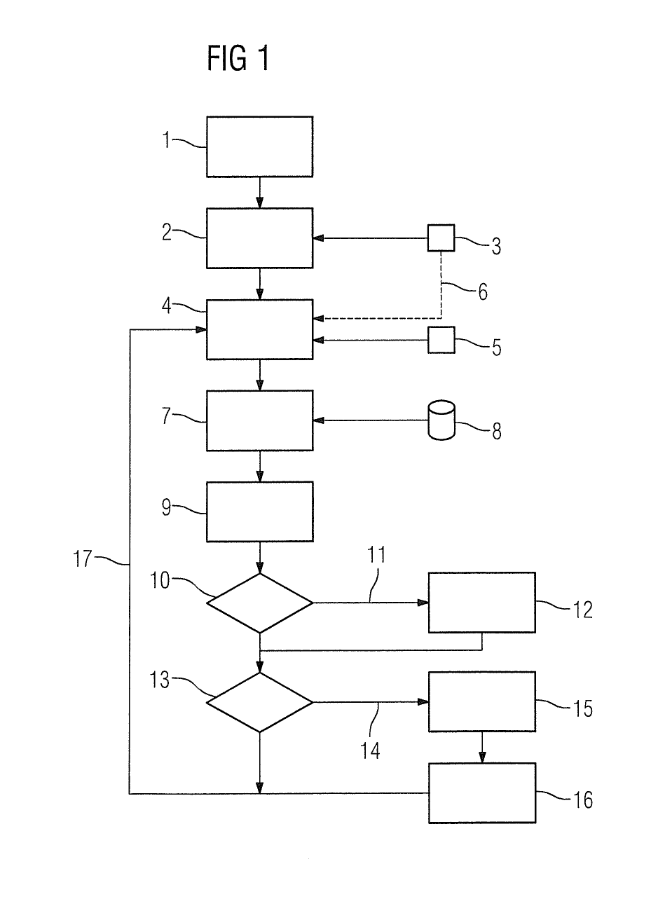

FIG. 1 shows a flowchart of an exemplary embodiment of the method according to the invention. This allows up-to-date assistive information regarding a medical apparatus (thus a medical technology apparatus) to be provided to an operator in a manner that is context-sensitive, intuitive and easily comprehensible to the operator. In general, a status of the medical apparatus can be transferred (as operating parameters describing the current operating status of said medical apparatus) via a wireless, bidirectional communication connection (here WLAN and/or Bluetooth) to a mobile auxiliary device (here a tablet computer, shortened to tablet), be evaluated there and be used to determine and at least partially display assistive information at said mobile auxiliary device. In order to conduct the evaluation and receive the information, a mobile application according to the invention is used, thus a computer program that is usable on different mobile auxiliary devices independent of platform. This is explained in detail in the following.

The mobile application is started in Step 1. In the event that it does not already exist—for example because both the mobile auxiliary device and the medical apparatuses are logged into a WLAN network—the wireless bidirectional communication connection is established in a known manner. The mobile application can be suitable for determining and at least partially displaying assistive information for multiple medical apparatuses. This means that it must initially be established for which at least one medical apparatus the operator presently requires assistive information.

This occurs in Step 2. The medical auxiliary device (and thus the mobile application) receives an identification information 3 either upon a query signal of said mobile application (thus of said mobile auxiliary device) or due to a cyclical broadcast by the medical apparatuses. The identification information 3 is suitable to uniquely identify the medical technology apparatus. It can consequently include a unique identifier, but also (for example) itemized information (for example a precise designation of the medical apparatus), a version of the medical apparatus and a serial number of the medical apparatus. The language in which the medical apparatus is presently being operated can also be determined from the identification information, such that the mobile application can correspondingly set its language and that of the assistive information (which can be provided in multiple languages).

Selection of the most probable medical apparatus to be attended to can now take place based on location information because (for example) it can be assumed that the medical apparatus nearest the mobile auxiliary device (or the multiple nearest medical apparatuses) should be considered. However, if multiple medical apparatuses with associated wireless communication connections are provided it is also possible to display a selection menu at the mobile auxiliary device, via which selection menu the operator can select the medical apparatus with regard to which he or she desires assistive information. The sorting can thereby likewise take place according to the spatial distance of the medical apparatuses and/or the quality of the communication connection.

In the method according to the invention, there are multiple possibilities of how and where the access to the assistive information that is determined should take in the later proceedings. If the assistive information should be provided as quickly and reliably as possible and there is enough storage space at the mobile auxiliary device, it is preferred to download a database with the assistive information for this medical apparatus or, respectively, a portion of a database with assistive information for multiple medical apparatuses (wherein the portion relates to the medical apparatus) at the point in time at which it is clear which medical apparatus should be attended to, and to store said database or portion of a database there at least for the time period of the activity of the mobile application. It is also possible to always retrieve the assistive information that is required from a database via an additional communication connection to a server (for example via the Internet), in particular in the event that storage space and otherwise data traffic should be spared. However, if a database or a portion of a database is downloaded, this also takes place in Step 2 after the medical apparatus has been identified.

The actual procedure to determine and display the assistive information begins in Step 4. At this point in time, the operating parameters 5 describing the current state of the medical apparatus are received. Particularly in the case of a broadcast, the operating parameters 5 can in principle always be received together with the identification information 3, as is indicated by the dashed arrow 6. A common data packet is then present, wherein the identification information additionally helps to select the correct operating parameters 5 given multiple medical apparatuses.

In Step 7, the operating parameters are then evaluated in order to determine context-sensitive, suitable assistive information. For example, the operating parameters themselves or selection parameters derived from the operating parameters can thereby be used in order to ascertain assistive information associated with the current status of the medical apparatus in the aforementioned database 8. Depending on whether the database 8 has been downloaded onto the mobile auxiliary device or whether a remote access to the database 8 exists, the assistive information is directly retrieved from the locally stored database 8 or downloaded via the additional communication connection to the server.

Such download and/or access processes can be logged if a billing or other evaluation should be conducted. Such usage information (which can then be determined by the mobile application itself or an additional mobile application) can relate to the determination of the assistive information, but also to their display. The usage time of the mobile application can also be the subject of a usage information. The usage information can be suitably evaluated in order to generate a billing information.

The evaluation of the usage information does not need to take place in the mobile auxiliary device; rather, a transfer to an evaluation server is preferred. In particular, usage information of different mobile auxiliary devices/mobile applications can then be evaluated there so that the use of the service provided by the invention can be correctly invoiced. Naturally, however, the assistive information can also be provided by a manufacturer at no cost, wherein such usage information can nevertheless reasonably be evaluated in order to correspondingly improve the service.

The assistive information can be composed of the most varied portions. It can include text components (possibly with embedded images) just as much as solitary images or even sound files that provide acoustic assistance. However, it can be advantageous for the assistive information to also include operating parameters for the medical apparatus, for example even entire measurement protocols in the manner of a “measurement protocol book”, which will be described in further detail in the following.

The at least partial display of the assistive information at the mobile auxiliary device then takes place in Step 9. An intelligent search function is thereby part of the mobile application, such that an operator can quickly locate the information relevant to him. It is also conceivable to implement menu structures in the assistive information so that an operator can quickly find [sic] to respond to a concrete question. The display of the assistive information thus takes place interactively so that, for example, images embedded in the text can also be highlighted and the like. This thus means that the operating parameters are used in order to make a pre-selection for the assistive information and/or the manner of their presentation in a context-sensitive manner so that detail information that cannot be derived from the operating parameters can be found as quickly as possible for the operator.

As noted, suggestions for operating parameters (in particular also entire operating protocols for the medical apparatus, for example measurement protocols or, respectively, organ programs for image acquisition devices) can also be included in the assistive information. Here the method offers the additional possibility to directly use these operating parameters at the medical apparatus. For this purpose, in Step 10 it is checked whether one or more operating parameters (or an entire operating parameter set, in particular an operating protocol) has been selected for use at the medical apparatus. If this is the case (Arrow 11), the corresponding operating parameters are transferred in Step 12 to the medical apparatus, where various possibilities for their further use are conceivable that can in particular take place cumulatively.

It is thus initially possible to provide the operating parameters as an output to a display device of the medical apparatus as a suggestion that can then be accepted by the operator. The operating parameters are then used to control the medical apparatus. Alternatively, however, it is also conceivable to directly use the operating parameters to control the medical apparatus without previously making a query, such that ultimately (at least for special cases) the medical apparatus can also be controlled via the medical auxiliary device.

The operating parameters or operating protocols can ultimately exist sorted according to medical circumstances in the assistive information. This enables the operator to easily identify and then select them. This also contributes to the already indicated realization of a measurement or, respectively, imaging protocol book that is available online.

An additional functionality that is realized via the mobile application is the possibility to likewise pose a context-related inquiry to at least one support employee. This is reviewed in Step 13. If a support request is present (Arrow 14), depending on the type of inquiry support information together with an associated inquiry information (ultimately the fully written out inquiry) is transmitted to a support computer associated with the support employee. Support information can thereby be status information related to the current status of the medical apparatus (for example including operating parameters indicating an error or the current situation in a workflow) but can also be result information related to a job result of the medical apparatus (for example, an acquired image data set which is sent with the inquiry information as to how the contrast can be increased).

If inquiries relate to the operation of the medical apparatus itself, it can be appropriate if the current operating status of the medical apparatus is precisely known to the support employee. It is accordingly also possible in Step 15 that, after the activation of the support function, the communication connection to the medical apparatus is used in order to transfer a command information, wherein the medical apparatus takes a current depiction of a display device of the medical apparatus as an image file (thus image data; “screenshot”) upon receipt of the command information. These image data are then transferred to the mobile auxiliary device, from where they can be further transmitted as auxiliary information. In this way it is particularly simple for a support employee to draw conclusions and suggest a course of action.

Before the support information is transmitted in Step 15, it is anonymized on the one hand, wherein on the other hand the transfer takes place in an encrypted manner so that security requirements are also satisfied. Known approaches can be used for this purpose.

The at least one support employee—possibly also an expert forum—handles the inquiry. Suitable response information is determined automatically or by the support employee and transmitted to the mobile auxiliary device in Step 16. There it is displayed to the operator so that said operator can receive the desired response to his inquiry.

Moreover, it is also possible that response information is not sent according to Step 16, but rather that a call from the support employee to the operator takes place, for example. This is particularly advantageously possible if a telephone number of the mobile auxiliary device (which has a telephone function) is automatically transferred with the support information, such contacting the operator is possible in a simple manner.

The method is continued (which means that, according to Arrow 17, the workflow proceeds again with Step 4) when new (thus in particular updated) operating parameters from the medical apparatus are present. The assistive information is thus always kept current. The method is also ended upon ending (closing or exiting) the mobile application. It is noted that, naturally, a function can also be realized in which it is possible to switch between available medical apparatuses.

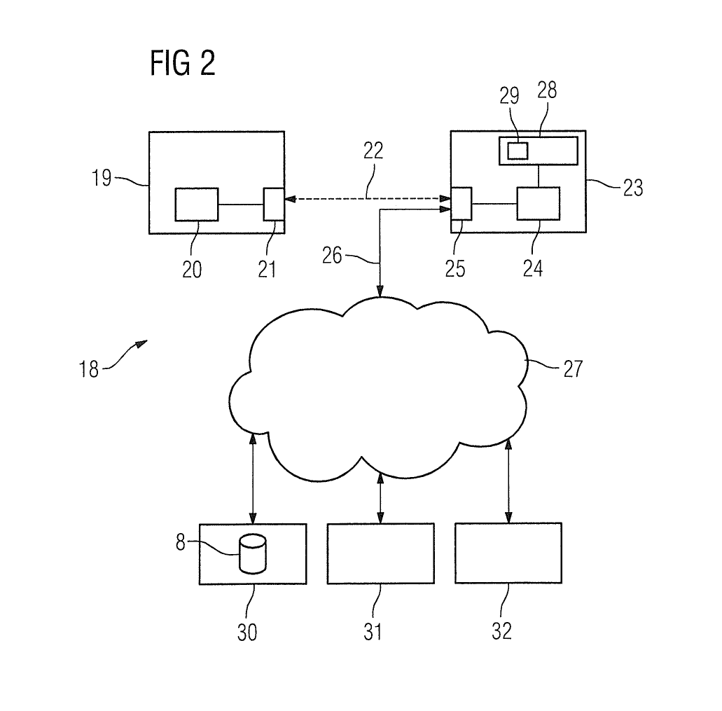

FIG. 2 schematically illustrates a system 18 according to the invention. The medical apparatus 19 can be a number of different devices that are known within the scope of medical technology. For example, the medical apparatus 19 can be an image acquisition device (in particular a magnetic resonance device, a CT device or the like), but it is also possible to use auxiliary devices or the like as medical apparatuses, for example contrast agent injectors and such devices.

The medical apparatus 19 has a control device 20 that controls the operation of said medical apparatus 19 but also supplies the necessary functions for the method according to the invention, consequently here provides an interface functionality together with the communication device 21. It is consequently a medical apparatus according to the invention. Operating parameters and identification data are provided in a defined format to the mobile auxiliary device 23 via the communication connection indicated by 22.

The mobile auxiliary device 23 (which can be a tablet or smartphone, for example) also has a corresponding control device 24 that, for example, can be formed by a CPU. Via a communication device 25, the mobile auxiliary device 23 can presently both communicate with the medical apparatus 19 and (Arrow 26) access a network 27, in particular the Internet and/or an intranet. The communication connection 22 can also be provided via the network 27 (which can include a WLAN, for example).

The medical auxiliary device 23 also has a storage device 28 in which the mobile application 29 can be stored, and it is also possible to store the assistive information or even the entire database 8 there, as described with regard to FIG. 1. The control device 24 executes the mobile application 29 according to the invention to realize the functions described with regard to FIG. 1.

Via the network 27, the mobile auxiliary device 23 can communicate with additional computers, for example here with a computer 30 fashioned as a server at which the database 8 can also be present. Additional devices to be named are the support computer 31 and an evaluation device 32 that receives usage data in order to evaluate them accordingly. It is noted that functionalities of the computers 30, 31 and/or 32 can naturally also be realized in joint computers so that, for example, the evaluation device 32 and the computer 30 can be realized as a single computer and the like.

The system 18 according to the invention is consequently designed to execute the method according to the invention, wherein the mobile application 29 represents an essential component because it is designed to evaluate the operating parameters (consequently to determine and display the assistive information). For this, the mobile application 29 must merely be executed on the mobile auxiliary device 23.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to determine and display assistive information related to a current operating status of a medical apparatus comprising:
from a medical apparatus, wirelessly transmitting operating parameters that describe a current operating status of the medical apparatus to a mobile auxiliary device;
at said mobile auxiliary device, executing a mobile application therein that uses the transmitted operating parameters to determine assistive information for said medical apparatus; and
displaying at least a portion of said assistive information at a display at said mobile auxiliary device operated by said mobile application.

2. A method as claimed in claim 1 comprising configuring said mobile application as a platform-independent mobile application that is usable in a plurality of different mobile auxiliary devices respectively from a plurality of different manufacturers.

3. A method as claimed in claim 1 comprising:
configuring said mobile application to determine and display said assistive information regarding a plurality of different medical apparatuses;
from said medical apparatus having said current operating status, transmitting, together with said operating parameters, identification information that identifies said medical apparatus; and
configuring said mobile application to set itself, upon receipt of said identification information, to determine and display said assistive information regarding the medical apparatus identified by said identification information.

4. A method as claimed in claim 3 comprising configuring said mobile application to request said identification application in a query signal sent from said mobile auxiliary application.

5. A method as claimed in claim 1 comprising:
during ongoing operation of said medical apparatus, transmitting any change in said operating parameters from said medical apparatus to said mobile auxiliary device; and
at said mobile auxiliary device, automatically updating said assistive information upon receiving modified operating parameters.

6. A method as claimed in claim 1 comprising:
storing said assistive information in a database; and
determining said assistive information at said mobile auxiliary device by, within said mobile application, using said operating parameters, or query parameters derived from said operating parameters, to access and retrieve said assistive information from said database.

7. A method as claimed in claim 6 comprising storing said assistive information in a database selected from the group consisting of a database in said mobile auxiliary device, a database located externally of said mobile auxiliary device.

8. A method as claimed in claim 7 comprising automatically loading said database into said mobile auxiliary device upon start-up of said mobile application in said mobile auxiliary device.

9. A method as claimed in claim 6 comprising:
configuring said mobile application to determine and display said assistive information regarding a plurality of different medical apparatuses;
from said medical apparatus having said current operating status, transmitting, together with said operating parameters, identification information that identifies said medical apparatus;
configuring said mobile application to set itself, upon receipt of said identification information, to determine and display said assistive information regarding the medical apparatus identified by said identification information;
in said database, storing assistive information for each of said plurality of different medical apparatuses; and
upon receipt of said identification information, downloading, to said mobile auxiliary device, only assistive information from said database related to the medical apparatus identified by said identification information.

10. A method as claimed in claim 7 comprising downloading only assistive information related to the current operating status of the medical apparatus to said mobile auxiliary device.

11. A method as claimed in claim 1 wherein said mobile application is configured to receive a user input that designates a portion of said assistive information to be displayed at said mobile auxiliary device, and displaying only the assistive information at said mobile auxiliary device that is designated by said input.

12. A method as claimed in claim 1 comprising configuring said mobile application to display, as said at least a portion of said assistive information, at least one further operating parameter for operating said medical apparatus.

13. A method as claimed in claim 12, comprising:
making a user entry into said mobile auxiliary device that selects said further operating parameter;
following said entry, transmitting said further parameter from said mobile auxiliary device to said medical apparatus; and
at said medical apparatus, making said further operating parameter available at a display of an operator terminal at the medical apparatus and (a) immediately beginning operation of said medical apparatus according to said further operating parameter, or (b) waiting for a user entry in said operating terminal that instructs said medical apparatus to operate to said further operating parameter.

14. A method as claimed in claim 12 wherein said assistive information comprises respective further operating parameters for different medical circumstances.

15. A method as claimed in claim 1 comprising:
configuring said mobile application to include a support function that is activatable by a user of the mobile auxiliary device;
configuring said support function to communicate with a support employee via a support transmission path that is different from a transmission path between said medical apparatus and said mobile auxiliary device; and
upon activation of said support function by the user of the mobile auxiliary device, transmitting said status information related to the current status of the medical apparatus, or result information describing an operational result achieved by said medical apparatus, to said support employee via said support communication path.

16. A method as claimed in claim 15 comprising:
configuring said support function to automatically transmit, upon activation of said support function by the user of said mobile auxiliary device, command information from the mobile auxiliary device to medical apparatus instructing said medical apparatus to transmit, to the mobile auxiliary device, image data showing a current presentation at a display device of the medical apparatus, as said status information or said result information, which is then transmitted to said support employee.

17. A method as claimed in claim 16 comprising configuring said support function to encrypt said status information and said result information before transmission to said support employee.

18. A method as claimed in claim 15 comprising transmitting response information from said support employee to said mobile auxiliary device and displaying said response information at said mobile auxiliary device.

19. A method as claimed in claim 1 comprising configuring said mobile auxiliary device as a device selected from the group consisting of mobile telephones, smart phones and computerized tablets.

20. A medical apparatus comprising:
a plurality of medical apparatus components that operate, in combination, according to operating parameters; and
an interface in communication with at least some of said plurality of components, said interface being configured to establish a wireless communication connection between said interface and a mobile auxiliary device and to wirelessly communicate said operating parameters, as a description of a current operating status of the medical apparatus, from the interface to the mobile auxiliary device via the communication connection.

21. A non-transitory, computer-readable data storage medium encoded with programming instructions, said programming instructions being configured as a mobile application and said storage medium being contained in a mobile auxiliary device and communicating with a processor of said mobile auxiliary device, said programming instructions causing said processor of said mobile auxiliary device to:
wirelessly receive operating parameters transmitted by a medical apparatus that describe a current operating status of the medical apparatus;
evaluate said operating parameters in said mobile application; and
in said mobile application, determine assistive information relating to operation of said medical apparatus and displaying at least a portion of said assistive information at a display at said mobile auxiliary device.

22. A medical system comprising:
a medical apparatus comprising a plurality of apparatus components that operate, in combination, according to operating parameters;
said apparatus comprising an interface configured to wirelessly transmit said operating parameters as a description of a current operating status of the medical apparatus to a mobile auxiliary device;
a mobile auxiliary device configured to execute a mobile application therein to receive and use the transmitted operating parameters to determine assistive information for said medical apparatus, said mobile auxiliary device comprising a display; and
said mobile application being configured to display at least a portion of said assistive information at said display of said mobile auxiliary device.
